(19) United States
(12) Patent Application Publication
(10) Pub. No.: US 2024/0306598 A1
(43) Pub. Date: Sep. 19, 2024

(54) KENNEL FOR DOGS

(71) Applicant: IP CAPITAL FUND S.C.A., Ville-Haute (LU)

(72) Inventor: Fabio LETTIERI, Ville-Haute (LU)

(73) Assignee: IP CAPITAL FUND S.C.A., Ville-Haute (LU)

(21) Appl. No.: 18/681,536

(22) PCT Filed: Jul. 14, 2022
(86) PCT No.: PCT/IB2022/056499

§ 371 Date: Feb. 6, 2024

(30) Foreign Application Priority Data
Aug. 6, 2021 (IT) 102021000021368

(51) Int. Cl. A01K 1/03; A01K 1/035; A01K 5/02; A01K 7/02; A01K 29/00

(52) U.S. Cl. CPC A01K 1/034; A01K 1/035; A01K 5/0291; A01K 7/025; A01K 7/027; A01K 29/005

(57) ABSTRACT

A kennel for dogs comprising at a least one perimeter wall covered at the top by at least one roof; at least one internal flooring defining, together with the perimeter wall, at least one housing area for at least one dog, the perimeter wall being provided with at least one access opening which the dog can pass through to gain access to the housing area; at least one monitoring system of the health status of the dog; at least one electronic processing and control unit operationally linked to the monitoring system.

### TECHNICAL FIELD

The present invention relates to a kennel for dogs.

### BACKGROUND ART

In its most traditional form, a kennel is a structure, usually made of wood, intended to define an area of housing habitable by at least one dog which is enclosed at the top by a roof and, at least partly, peripherally by a perimeter wall.

Although the kennel is typically imagined as the bed used by the dog for sleeping, it is good to keep in mind that it represents a place for the animal to carry out many other activities besides resting; one among them, for example, guarding the owner's house, protecting it from thieves and prowlers.

Not only that, but the kennel is also often the place arranged for the dog to consume the food allocated to them by their owners who, for this purpose, fill at least partly one or more bowls that they arrange in the proximity of the kennel itself.

This causes the dog to be enticed to stay inside the kennel even to eat its own meal and/or drink.

It appears, therefore, evident that much of the dog's life is spent inside its kennel.

That said, there is nowadays an increasing need for dog owners to monitor their pet's health status more closely.

Scrupulous monitoring, in fact, makes it possible to become aware of one's animal's biophysical parameters, its growth over time and other relevant parameters still.

In addition, monitoring the animal's health status also enables early identification of whether it has contracted diseases, thus preventing complications of the same.

In this case, in fact, the appropriate veterinarian can be notified to provide treatment for the dog quickly.

### DESCRIPTION OF THE INVENTION

The main aim of the present invention is to devise a kennel for the dogs which enables dog owners to monitor the health status of their pet efficiently and easily.

Another object of the present invention is to devise a kennel for dogs which can overcome the aforementioned drawbacks of the prior art within the framework of a simple, rational, easy and effective to use as well as affordable solution.

The aforementioned objects are achieved by this kennel for dogs having the characteristics of claim 1.

### BRIEF DESCRIPTION OF THE DRAWINGS

Other characteristics and advantages of the present invention will become more apparent from the description of a preferred, but not exclusive, embodiment of a kennel for dogs, illustrated by way of an indicative, yet non-limiting example in the attached tables of drawings in which:

FIG. 1 is an axonometric view of the kennel according to the invention;

FIG. 2 a rear axonometric view of the kennel according to the invention;

FIG. 3 is a schematic view of the operation of the kennel according to the invention;

FIG. 4 is a schematic view of the operation of the automatic kennel watering system according to the invention;

FIG. 5 is a schematic view of the operation of the automatic kennel feeding system according to the invention.

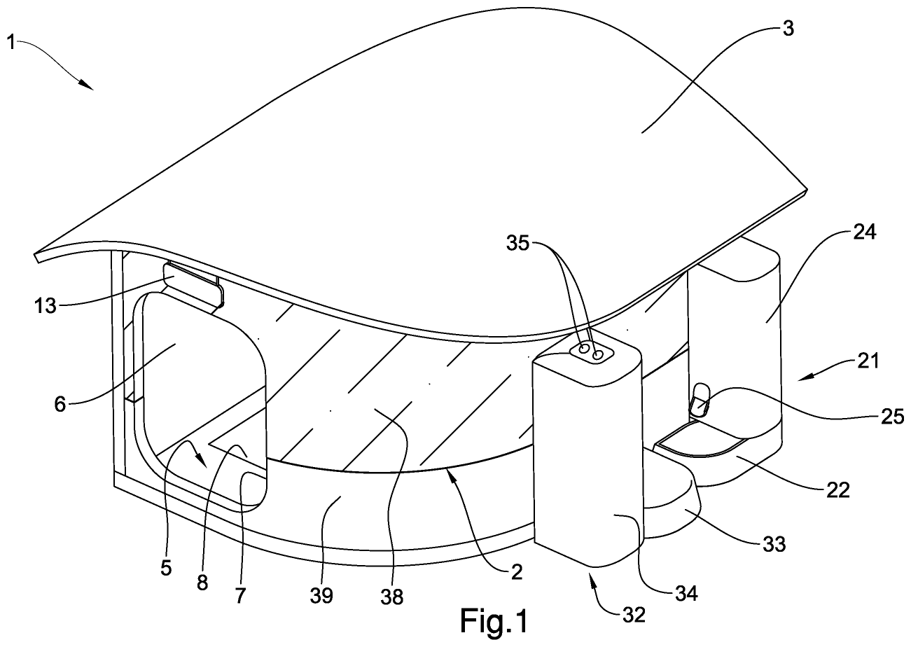

### EMBODIMENTS OF THE INVENTION

With particular reference to these figures, reference numeral 1 globally indicates a kennel for dogs.

The kennel 1 for dogs comprises at least one perimeter wall 2 covered at the top by at least one roof 3.

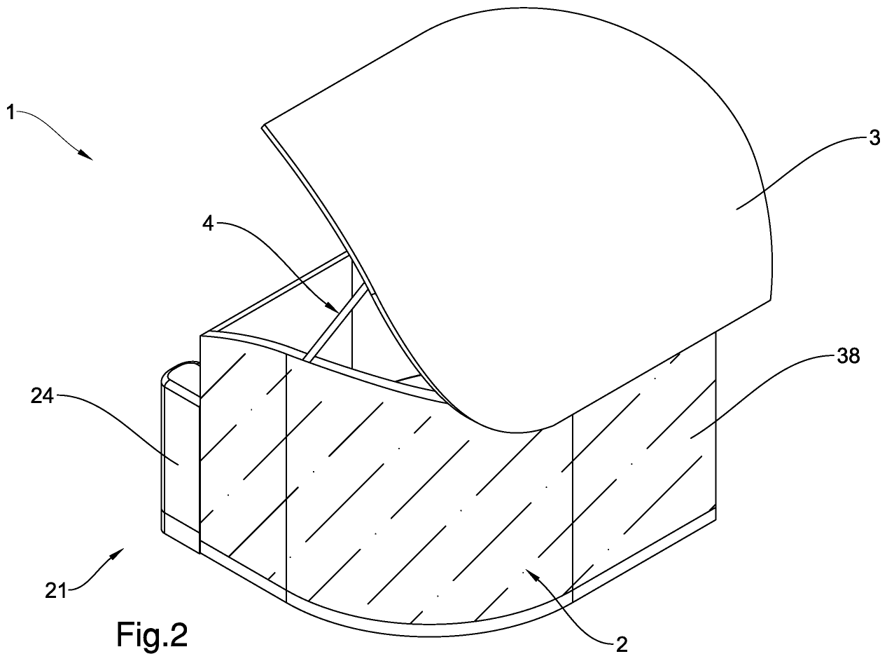

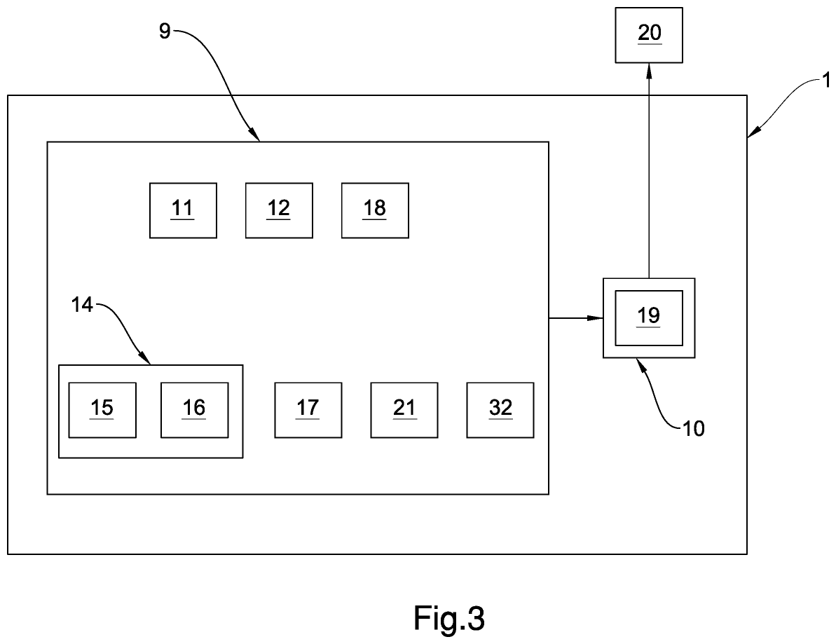

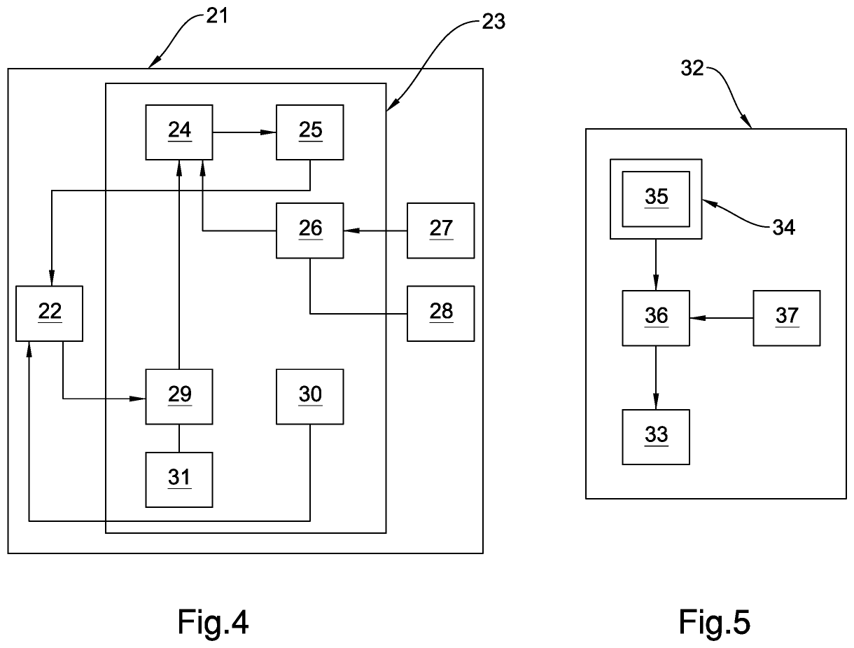

As visible in FIG. 2, the roof 3 comprises manual lifting means 4 associated with the perimeter wall 2 and adapted to lift the roof 3 with respect to the perimeter wall 2.

In turn, the manual lifting means 4 comprise at least one supporting body 4 of the roof 3 when the latter is lifted with respect to the perimeter wall 2.

In fact, the latter expedient allows preventing the roof 3 from rearranging itself in contact with the perimeter wall 2 by closing it up.

Specifically, the supporting body 4 is positioned, in use, between the roof 3 and the perimeter wall 2.

Again, the supporting body 4 is of the inextensible type.

In other words, the supporting body prevents the roof 3 from closing up by interposing itself between the latter and the perimeter wall 2.

For example, the supporting body 4 is of the type of a rod.

In particular, the supporting body 4 is made of a metal material.

The kennel 1 also comprises at least one internal flooring 5 defining, together with the perimeter wall 2, at least one housing area 6 for at least one dog, the perimeter wall 2 being provided with at least one access opening 7 which the dog can pass through to gain access to the housing area 6.

It is, therefore, easy to appreciate that the special expedient of providing manual lifting means 4 allows not only for the cleaning of the housing area 6 to be carried out adequately, quickly and easily, but also for effective monitoring of whether the dog has experienced symptoms that can be linked to one or more diseases within the housing area 6 such as, e.g., vomiting, diarrhea, excessive shedding of hair or other non-physiological conditions.

Advantageously, the internal flooring 5 comprises at least one heating body 8 which can be used by the dog to warm itself, e.g. to warm itself during winter.

In this regard, the heating body 8 is electrically powered.

In this way, in fact, the heating body 8 can generate heat by Joule effect and heat itself.

In accordance with the preferred embodiment, the heating body 8 is of the type of an electrically powered folding mat.

Advantageously, the kennel 1 comprises a plurality of movement elements, not shown in the figures, associated below with the perimeter wall 2.

Specifically, the movement elements are of the wheel type.

This allows the kennel 1 to be easily moved to different locations from each other.

According to the invention, the kennel 1 comprises:
at least one monitoring system 9 of the health status of the dog;
at least one electronic processing and control unit 10 operationally linked to the monitoring system 9.

In accordance with a preferred embodiment, the kennel 1 comprises at least one insertion compartment for the electronic processing and control unit 10.

Specifically, the electronic processing and control unit 10 is associated with the insertion compartment due to at least one linking element positioned between the two.

For example, the linking element is of the type of a screw connector.

In addition, the insertion compartment is made on the roof 3.

Different placements of the insertion compartment such as, e.g., on the perimeter wall 2 cannot however be ruled out.

In addition, the electronic processing and control unit 10 is electrically powered by means of at least one electrical cable fitted in at least one sheath made up at least partly of an anti-bite material.

It is specified that, in the substance of this disclosure, by “anti-bite” is meant to describe a material provided with chemical and physical characteristics that make it resistant to incisions and cuts caused, e.g., by dog bites.

By doing so, in fact, possible malfunctions of the electronic processing and control unit 10 due to dog bites to the electric cable can be averted.

Conveniently, the monitoring system 9 comprises weight monitoring means 11 of the dog configured to send to the electronic processing and control unit 10 at least one signal relating to the weight of the dog, the electronic processing and control unit 10 being configured to determine the presence of the dog inside the housing area 6 depending on the reception of the signal relating to the weight of the dog.

Specifically, the weight monitoring means 11 comprise at least one weight sensor 11 associated with the internal flooring 5.

In the present case, the weight sensors 11 are of the type of load cells.

In detail, the weight monitoring means 11 comprise four weight sensors 11 arranged in the proximity of the four corners of the internal flooring 5.

In this way, in fact, it is possible to monitor the weight of the dog whenever it lodges in the housing area 6.

This also makes it possible to determine, through repeated monitoring over time, whether the dog is gaining weight, losing weight or maintaining weight.

Advantageously, the monitoring system 9 comprises temperature monitoring means 12 adapted to send to the electronic processing and control unit 10 at least one signal relating to temperature.

Specifically, the temperature monitoring means 12 comprise at least one temperature sensor 12 associated with the internal flooring 5.

In this case, the temperature sensor 12 is of the type of a pyroelectric sensor.

More specifically, the temperature monitoring means 12 comprise at least one array of the temperature sensors 12 which are distributed spaced apart from each other.

To be precise, the temperature monitoring means 12 comprise an array of sixty-four temperature sensors 12 which are distributed spaced apart from each other.

In addition, the temperature monitoring means 12 are configured to operate continuously over time.

This means, therefore, that the temperature monitoring means 12 monitor the internal temperature of the housing area 6 even when the dog is not present therein.

In this regard, the electronic processing and control unit 10 is programmed with at least one reference temperature value and is configured to compare the signal relating to temperature with the reference temperature value to determine, depending on this comparison, the presence of the dog inside the housing area 6.

Conveniently, the reference temperature value is higher than the average internal temperature of the housing area 6 and, at the same time, lower than the dog's average body temperature.

Emphasis is placed on the fact that providing for temperature monitoring means 12 allows the dog's body temperature to be monitored and thus determine whether the dog is feverish.

Not only that, but the fact of providing for temperature monitoring means 12 also allows the frequency with which the dog enters and leaves the housing area 6 and the time it spends therein to be monitored.

Conveniently, the monitoring system 9 comprises at least one indicator light 13 of the presence of the dog associated with the perimeter wall 2 and operationally connected to the electronic processing and control unit 10 to light up, indicating the presence of the dog inside the housing area 6, as a result of the determination by the electronic processing and control unit 10 of the presence of the dog.

In other words, the electronic processing and control unit 10 activates the indicator light 13 when it receives the signal relating to weight and when the signal relating to temperature is greater than the reference temperature.

In this regard, the indicator light 13 is of the type of a luminous siren, of one or more LEDs or other type of indicator light which allows, through its own illumination, to signal the presence of the dog inside the housing area 6.

Preferably, the monitoring system 9 comprises visual monitoring means 14 of the dog which are adapted to capture images of the housing area 6 and to send at least one signal to the electronic processing and control unit 10 containing the images when obtaining at least one video signal.

Specifically, the visual monitoring means 14 comprise at least one camera 14.

Conveniently, the camera 14 comprises lighting means 15.

Specifically, the lighting means 15 are of the infrared sensor type.

Advantageously, the lighting means 15 comprise at least one outdoor brightness sensor configured to compare outdoor brightness with a preset brightness value.

In this regard, the lighting means 15 are configured to activate automatically if the outdoor brightness is lower than the preset value in the outdoor brightness sensor.

It is emphasized, in this regard, that the fact of providing lighting means 15 allows for the efficient use of the visual monitoring means 14 even in low-visibility situations, such as e.g. at night.

In addition, the visual monitoring means 14 shall comprise at least one wireless linking module 16 to a dedicated computer portal for sharing the signal acquired by the visual monitoring means 14 with one or more vets.

This advantageously makes it possible to intervene quickly with medical treatment should the signal acquired by the visual monitoring means 14 suggest that the dog is suffering from one or more diseases.

Conveniently, the monitoring system 9 comprises gas monitoring means 17 of the gases contained inside the housing area 6 which are adapted to send to the electronic processing and control unit 10 at least one signal relating to the concentration and to the type of the gases, the electronic processing and control unit 10 being configured to compare the values with corresponding reference values to determine, depending on this comparison, whether the dog is affected by one or more pathologies.

In addition, the monitoring system 9 comprises at least one sterilizing device 18 configured to sterilize the housing area 6 when the dog is not present therein.

This allows, in fact, the gas monitoring means 17 to carry out more accurate monitoring, avoiding the accumulation of gases inside the housing area thus distorting the measurements taken.

Specifically, the sterilizing device 18 is of the type of a UVC lamp or a UVB lamp.

In other words, the sterilizing device 18 sterilizes the housing area 6 due to the germicidal action exerted by ultraviolet radiation.

Advantageously, the electronic processing and control unit 10 comprises at least one remote connection module 19 to at least one digital processor 20 configured to display in real time at least one of: the signal relating to weight, the signal relating to temperature, the video signal and the signal relating to gas concentration and type.

This, therefore, allows monitoring of the dog's health status over time.

In addition, the monitoring system 9 comprises at least one automatic watering system 21 for the dog, comprising:
at least one bowl 22 designed to hold water for the dog to drink;
at least one hydraulic assembly 23 comprising:
   at least one water tank 24;
   at least one main pipe 25 associated with the tank 24 and connected in a fluid-operated manner to the bowl 22 for at least partly filling the latter with water;
   at least one main pumping device 26 operationally connected to the tank 24 for pumping water from the latter to the bowl 22;
at least one activation sensor 27 operationally connected to the main pumping device 26 and configured to detect the presence of the dog in the proximity of the bowl 22 and to activate, consequently to the detection, the main pumping device 26.

In detail, the main pumping device 26 allows pumping substantially constant flow rates of water from the tank 24 to the bowl 22.

This makes it possible, conveniently, to monitor the dog's water consumption over time by measuring, at regular intervals, the amount of water pumped externally to the tank 24, e.g., by measuring it daily.

In addition, the automatic watering system 21 comprises at least one cooling device 28 associated with the tank 24 and adapted to reduce the temperature of the water contained inside the latter.

In this case, the cooling device 28 is of the Peltier cell type.

This keeps the water to be dispensed into the bowl 22 cool even in particularly adverse weather conditions such as, e.g., during summer.

In addition, the hydraulic assembly 23 comprises:
at least one secondary pipe 29 associated with the bowl 22 and connected in a fluid-operated manner to the tank 24 for the reintroduction inside the latter of the residual water in the bowl 22;
at least one secondary pumping device 30 operationally connected to the bowl 22 for pumping water from the latter to the tank 24.

In this regard, the activation sensor 27 is operationally connected to the secondary pumping device 30 and is configured to activate, consequently to the interruption of the detection of the presence of the dog, the secondary pumping device 30.

This, therefore, allows the residual water in the bowl 22 to be recovered and reintroduced into the tank 24.

Advantageously, the hydraulic assembly 23 comprises at least one water filtration device 31 associated with the secondary pipe 29.

In this way, in fact, the water reintroduced into the tank is filtered from dust and/or foreign bodies and is again drinkable by the dog.

Conveniently, the monitoring system 9 comprises at least one automatic feeding system 32 for the dog comprising:
at least one cup 33 intended to contain food for feeding the dog;
at least one container 34 of food.

In this regard, the container 34 is provided with at least one food inlet hole 35 for the at least partial filling of the container itself.

Specifically, the container 34 is provided with two inlet holes 35.

Conveniently, the inlet holes 35 are arranged on top of the container 34.

The automatic feeding system 32 then comprises at least one transport element 36 of the food from the container 34 to the cup 33.

Conveniently, the transport element 36 is of the type of a worm screw.

It cannot however be ruled out that the transport elements 36 be of a different type such as, e.g., of a wedge dispenser formed, that is, by a body rotating on itself and provided with barriers defining, between them, hollow sectors that can be filled by the food for the transport of the latter externally to the container 34.

In other words, the transport element 36 is shaped so as to transport food externally to the container 34.

In all cases, the transport element 36 is shaped so as to carry substantially equal amounts of food.

This makes it possible, similarly to the automatic watering system 21, to monitor the dog's food consumption over time by measuring, e.g., whether the dog consumes its meal regularly.

In addition, the automatic feeding system 32 comprises at least one timed sensor 37 operationally connected to the transport element 36 and programmed with at least one preset time range.

Specifically, the timed sensor 37 is configured to actuate the transport element 36 at regular time ranges substantially coinciding with the preset time range.

This allows food to be dispensed into the cup 33 at predetermined times and thus prevent the dog from consuming excessive amounts of food.

According to a further aspect, the present invention also relates to a kennel 1 for dogs in which the perimeter wall 2 is provided with at least one portion 38 made, at least partly, of a transparent material.

It is specified, within the scope of this disclosure, that “transparent” is intended to define a material that can be traversed throughout its thickness by light radiation, thus allowing vision through it.

In fact, this expedient allows the dog to watch over the area around the kennel 1 while staying inside the housing area 6.

Not only that, but in this way the dog can survey the outside area significantly more effectively than would be the case in kennels of known type, in which the dog has a more limited field of vision.

In addition, in kennels of known type, the animal is forced to face externally to the housing area 6 to watch over the outside area, exposing itself to the weather elements such as, e.g., rain and snow.

It is specified, regarding the present aspect, that the components of the present invention described above are not discussed again.

In other words, the components common to both aspects are numbered with the same reference number and are not described further on.

This means that the previously stated considerations regarding common components remain valid for this further aspect of the invention.

Advantageously, the perimeter wall 2 is provided with at least one protection band 39 which is adapted to protect the portion 38 from the scratches of the dog. In detail, the protection band 39 is made, at least partly, of opaque material.

Within the scope of this disclosure it is specified that “opaque” is intended to define a material that is generically non-transparent and such, that is, as to absorb or refract light radiation.

In particular, the portion 38 is located at a substantially higher level than the level of the protection band 39.

In this way, in fact, the portion 38 is protected from dog scratches.

It has in practice been ascertained that the described invention achieves the intended objects.

In particular, the fact is emphasized that the special expedient of providing for a dog health monitoring system enables dog owners to monitor the health status of their pet efficiently and easily.

1) A kennel for dogs, the kennel comprising:
at least one perimeter wall covered at a top by at least one roof;
at least one internal flooring defining, together with said at least one perimeter wall, at least one housing area for at least one dog, said at least one perimeter wall being provided with at least one access opening which the at least one dog can pass through to gain access to said at least one housing area;
at least one monitoring system of a health status of the at least one dog; and
at least one electronic processing and control unit operationally linked to said at least one monitoring system.

2) The kennel according to claim 1, wherein said at least one monitoring system comprises weight monitoring means of the at least one dog configured to send to said at least one electronic processing and control unit at least one signal relating to a weight of the dog, said at least one electronic processing and control unit being configured to determine a presence of the at least one dog inside said at least one housing area depending on the reception of said signal relating to the weight of the at least one dog.

3) The kennel according to claim 2, wherein said weight monitoring means comprise at least one weight sensor associated with said at least one internal flooring.

4) the kennel according to claim 3, wherein said weight monitoring means comprise four weight sensors arranged in proximity of four corners of said at least one internal flooring.

5) The kennel according to claim 2, wherein said at least one monitoring system comprises temperature monitoring means adapted to send to said at least one electronic processing and control unit at least one signal relating to temperature, the at least one electronic processing and control unit being programmed with at least one reference temperature value and being configured to compare said signal relating to temperature with said at least one reference temperature value to determine, depending on said comparison, the presence of the at least one dog inside said housing area.

6) The kennel according to claim 5, wherein said temperature monitoring means comprise at least one temperature sensor associated with said at least one internal flooring.

7) The kennel according to claim 6, wherein said at least one temperature monitoring means comprise at least one array of said temperature sensors distributed spaced apart from each other.

8) The kennel according to said at least one monitoring system comprises at least one indicator light of the presence of the dog associated with said at least one perimeter wall and operationally connected to said at least one electronic processing and control unit to light up, indicating the presence of the dog inside said at least one housing area, as a result of said determination by said at least one electronic processing and control unit of the presence of the at least one dog.

9) the kennel according to claim 1, wherein said at least one monitoring system comprises visual monitoring means of the at least one dog which are adapted to capture images of said at least one housing area and to send at least one signal to said at least one electronic processing and control unit containing said images when obtaining at least one video signal.

10) The kennel according to claim 9, wherein said visual monitoring means comprise at least one camera.

11) The kennel according to claim 1, wherein said at least one monitoring system comprises gas monitoring means of gases contained inside said at least one housing area which are adapted to send to said at least one electronic processing and control unit at least one signal relating to concentration and to a type(s) of said gases, said at least one electronic processing and control unit being configured to compare values with corresponding reference values in order to determine, depending on said comparison, whether the at least one dog is affected by one or more pathologies.

12) The kennel according to claim 11, wherein said at least one electronic processing and control unit comprises at least one remote connection module to at least one digital processor configured to display in real time at least one of: said signal relating to weight, said signal relating to temperature, said video signal and said signal relating to the gas concentration and said type(s).

13) The kennel according to claim 2, wherein said at least one monitoring system comprises at least one automatic watering system for the at least one dog. comprising:
at least one bowl designed to hold water for the at least one dog to drink;
at least one hydraulic assembly comprising:
at least one water tank;
at least one main pipe associated with said at least one water tank and connected in a fluid-operated manner to said at least one bowl for at least partly filling said at least one bowl with water;
at least one main pumping device operationally connected to said at least one water tank for pumping water from said at least one water tank to said at least one bowl; and
at least one activation sensor operationally connected to said at least one main pumping device and configured to detect the presence of the at least one dog in a proximity of said at least one bowl and to activate, consequently to said detection, said at least one main pumping device.

14) The kennel according to claim 13, wherein said at least one automatic watering system comprises at least one cooling device associated with said at least one water tank and adapted to reduce a temperature of the water contained inside said at least one water tank.

15) The kennel according to claim 14, wherein said at least one hydraulic assembly comprises:
at least one secondary pipe associated with said at least one bowl and connected in a fluid-operated manner to said at least one water tank for reintroduction inside said at least one water tank of the residual water in said at least one bowl; and
at least one secondary pumping device operationally connected to said at least one bowl for pumping said water from said at least one bowl to said at least one water tank.

16) The kennel according to claim 15, wherein said at least one activation sensor is operationally connected to said at least one secondary pumping device and is configured to activate, consequently to an interruption of the detection of the presence of the at least one dog, said at least one secondary pumping device.

17) The kennel according to claim 16, wherein said at least one hydraulic assembly comprises at least one water filtration device associated with said at least one secondary pipe.

18) The kennel according to claim 1, wherein said monitoring system comprises at least one automatic feeding system for the at least one dog, comprising:
at least one cup intended to contain food for feeding the at least one dog;
at least one container of food;
at least one transport element of the food from said at least one container to said at least one cup;
at least one timed sensor operationally connected to said at least one transport element and programmed with at least one preset time range, said at least one timed sensor being configured to actuate said at least one transport element at regular time ranges substantially coinciding with said at least one preset time range.

19) A kennel for dogs, the kennel comprising:
at least one perimeter wall covered at a top by at least one roof; and
at least one internal flooring defining, together with said at least one perimeter wall, at least one housing area for at least one dog, said at least one perimeter wall being provided with at least one access opening which the at least one dog can pass through to gain access to said at least one housing area, wherein
said at least one perimeter wall is provided with at least one portion made, at least partly, of transparent material.

20) The kennel according to claim 19, wherein said at least one perimeter wall is provided with at least one protection band which is adapted to protect said at least one portion from scratches of the at least one dog.

21) The kennel according to claim 20, wherein said at least one portion is located at a substantially higher level than the level of said at least one protection band.
